(19) United States
(12) Patent Application Publication
(10) Pub. No.: US 2014/0295547 A1
(43) Pub. Date: Oct. 2, 2014

(54) METHOD TO CONSTRUCT IN-VITRO HUMAN BLOOD BRAIN BARRIER MODEL

(71) Applicant: National Chung Cheng University, Chia-Yi (TW)

(72) Inventors: Yung-Chih KUO, Chia-Yi (TW); Chin-Lung LEE, Chia-Yi (TW)

(73) Assignee: National Chung Cheng University, Chia-Yi (TW)

(21) Appl. No.: 13/974,561

(22) Filed: Aug. 23, 2013

(30) Foreign Application Priority Data
Mar. 29, 2013 (TW) 102111425

(51) Int. Cl. C12N 5/071

(52) U.S. Cl. CPC C12N 5/0697
USPC 435/368

(57) ABSTRACT

A method to construct an in-vitro human blood brain barrier (BBB) model is disclosed, which comprises steps: attaching suspension liquids of human brain vascular pericytes (HBVPs) and human astrocytes (HAs) by a ratio of 1:1, 1:2, or 1:6 to a bottom surface of a filter membrane of a culture dish to plant HBVPs and HAs on the bottom surface; filling a suspension liquid of human brain microvascular endothelial cells (HBMECs) into a top surface of the filter membrane to plant HBMECs on the top surface; placing the culture dish in a well plate containing a culture medium, and placing the well plate in a carbon-dioxide incubator; replacing the culture medium with a condition medium; and replacing the condition medium once daily for a plurality of days. Thereby is constructed an in-vitro human BBB model of high medical research availability.

### BACKGROUND OF THE INVENTION

1. Field of the Invention

The present invention relates to a model construction technology, particularly to a method to construct an in-vitro human blood brain barrier model,

2. Description of the Related Art

The blood brain barrier (BBB) is a filter selectively retarding some materials from entering the brain, functioning like a brain health guard. While a person catches a cold, the blood brain barrier would securely protect the brain from invasion of viruses or bacteria lest viruses or bacteria cause meningitis. The blood brain barrier of a healthy person can normally protect the brain strictly. If the brain of a person should be infected by viruses or bacteria, it indicates that the person has some problems in health and needs appropriate rest or even medical inspection.

At present, there have been technologies co-culturing rat brain microvascular endothelial cells and either of rat brain vascular pericytes or rat astrocytes, or co-culturing all of them to construct a BBB model. A BBB model, which simultaneously contains astrocytes and brain vascular pericytes in addition to the brain microvascular endothelial cells, has higher transendothelial electrical resistance (TEER) and lower permeability and is more likely to simulate the BBB effectively. The BBB model co-culturing the abovementioned three types of cells has been used to test drugs. The coverage ratio of brain vascular pericytes to brain microvascular endothelial cells varies with the physiological and pathological statuses. However, the conventional technologies do not pay attention to the coverage ratio of brain vascular pericytes to brain microvascular endothelial cells. Therefore, the conventional technologies lack a. BBB model considering the real physiological status.

Accordingly, the present invention proposes a method to construct an in-vitro human BBB model to solve the abovementioned problem.

### SUMMARY OF THE INVENTION

The primary objective of the present invention is to provide a method to construct an in-vitro human BBB model, wherein suspension liquids respectively containing human brain vascular pericytes (HBVPs) and human astrocytes (HAs) by different ratios are used to construct in-vitro BBB models, which can effectively simulate the in-vivo BBB and function as a test platform for various brain drugs and thus have high medical research availability.

To achieve the abovementioned objective, the present invention proposes a method to construct an in-vitro human BBB model, which comprises steps: attaching a suspension liquid of HBVPs and a suspension liquid of HAs by a ratio of 1:1, 1:2, or 1:6 onto the bottom surface of a filter membrane of a culture dish to plant HBVPs and HAs on the bottom surface; filling human brain microvascular endothelial cells (HBMECs) into the top surface of the filter membrane to plant HBMECs on the top surface; placing the culture dishes in a well plate containing a culture medium, letting the liquid level of the culture dishes be as high as the liquid level of the culture medium, and placing the well plate in a carbon-dioxide incubator to culture the cells; replacing the culture medium with a condition medium once HBMECs, HBVPs and HAs have occupied 80% of the filter membrane; and replacing the condition medium daily for several days.

Below, embodiments are described in detail in cooperation with drawings to make easily understood the technical contents, characteristics and accomplishments of the present invention.

### BRIEF DESCRIPTION OF THE DRAWINGS

FIG. 1 shows a flowchart of a method to construct an in-vitro human BBB model according to one embodiment of the present invention; and

FIG. 2 schematically shows that the suspension liquids of HBVPs and HAs are planted in a culture dish according to one embodiment of the present invention.

### DETAILED DESCRIPTION OF THE INVENTION

After subculture, HBMECs, HBVPs and HAs are further cultured in a porous filter membrane of a culture dish, such as a transwell. HBVPs and HAs are cultured on the bottom surface of the porous filter membrane, and HBMECs are cultured on the top surface of the porous filter membrane. After culture, HBMECs interconnect closely to form tight junctions (TJs) functioning as BBB. In the present invention, HBMECs are modulated by HBVPs and HAs to improve the integrity of the in-vitro BBB model and increase the activity of P-glycoprotein (P-gp).

The present invention uses HBMECs and different ratios of HBVPs and HAs to construct a BBB model and measures TEER and permeability of propidium iodide (PI) to evaluate the integrity and performance of the BBB model. The present invention also uses a fluorescent dye Calcein to detect the activity of P-gp. The present invention also detects the concentrations of the transforming growth factor-β1 (TGF-β1), the matrix. metalloproteinases (MMP-9), and the vascular endothelial growth factor (VEGF), which influence the integrity of the BBB model. The experimental results show that the BBB model with the ratio of HBVPs to HAs=1:2 has a higher concentration of TGF-β1 and lower concentrations of MMP-9 and VEGF than the other BBB models. It suggests that an in-vitro BBB model matching the in-vivo coverage ratio is more representative and medical-research available and can function as a test platform of brain drugs.

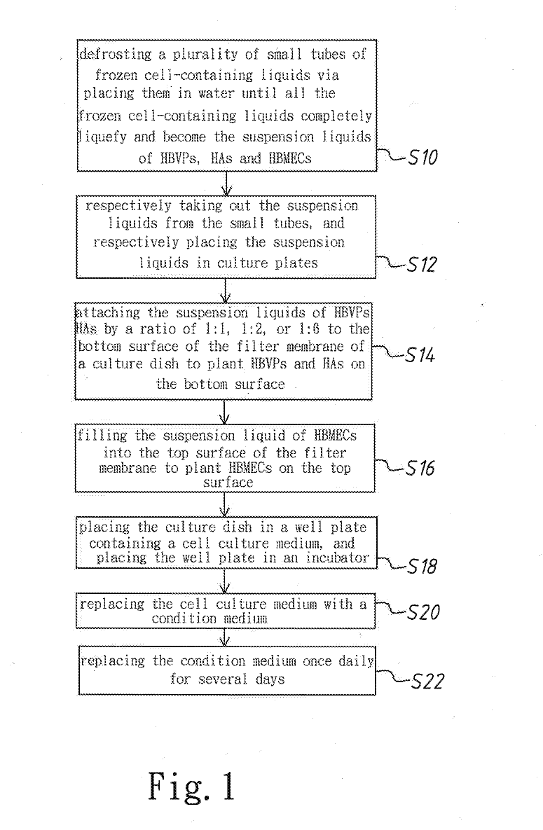

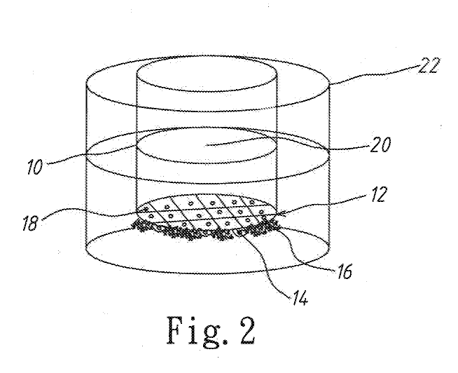

Refer to FIG. 1 and FIG. 2. FIG. 1 shows a flowchart of a method to construct an in-vitro BBB model according to one embodiment of the present invention. FIG. 2 schematically shows that the suspension liquids of HBVPs and HAs are planted in a culture dish according to one embodiment of the present invention. In Step S10, defrost a plurality of small tubes of frozen cell-containing liquids via placing them in water having a temperature of 37° C. for 1 minute until all the frozen cell-containing liquids completely liquefy and become the suspension liquids of HBVPs, HAs and HBMECs. In Step S12, respectively suck the suspension liquids of HBVPs, HAs and HBMECs from the small tubes, respectively place the suspension liquids in pretreated culture plates, and culture the suspension liquids in a carbon-dioxide incubator having a temperature of 37° C. and a relative humidity of 95%. In Step S14, provide a pretreated culture dish 10 having a filter membrane 12 made of PET (polyethylene terephthalate); flip over the filter membrane 12; attach the suspension liquids of HBVPs and HAs by a ratio of 1:1, 1:2, or 1:6 to the bottom surface of the filter membrane 12 of the culture dish 10 to plant HBVPs 14 and HAs 16 on the bottom surface, wherein the time for attaching is 1 hour and the total planting density of HBVPs 14 and HAs 16 is 4×10⁵ cells/cm². In Step S16, fill the suspension liquid of HBMECs into the top surface of the filter membrane 12 to plant HBMECs 18 on the top surface, wherein the planting density of HBMECs 18 is 4×10⁵ cell/cm². In Step S18, place the culture dish 10 in a well plate 22 containing a cell culture medium 20, and place the well plate 22 in a carbon dioxide incubator having a temperature of 37° C. and a relative humidity of 95% to culture the cells, wherein the liquid level of the culture dish 10 is as high as the liquid level of the cell culture medium 20, and wherein the cell culture medium 20 contains an endothelial cell medium, a pericytes culture medium and an astrocyte medium. Once HBVPs 14, HAs 16 and HBMECs 18 have occupied 80% of the filter membrane 12, the process proceeds to Step S20. In Step S20, replace the cell culture medium 20 with a condition medium, and keep on culturing the cells in the abovementioned carbon-dioxide incubator. The condition medium may be a pericyte-condition medium (PCM₁) having been used for 1 day, a pericyte-condition medium (PCM₂) having been used for 2 days, an astrocyte-condition medium (ACM₁) having been used for 1 day, an astrocyte-condition medium (ACM₂) having been used for 2 day, or a condition medium containing PCM₂ and ACM₂ by a ratio of 1:1. In Step S22, replace the condition medium each day, and repeat it for several days. In one embodiment, daily medium replacement is repeated for 7 days.

If the suspension liquids of HBVPs, HAs and HBMECs are readily available, Step S10 and Step S12 in the abovementioned process are unnecessary, and the process can start from Step S14 directly.

The present invention particularly pays attention to the coverage ratio and interaction of cells. It is found: in the case that HBMECs are co-cultured with HBVPs and HAS by a ratio of 1:2 and the co-culture is further cultured in a condition medium containing PCM and ACM by a ratio of 1:1 for 7 days, TEER is increased to as high as 319±16.67 Ω×cm², and the permeability of propidium iodide is decreased to only 39% of that of a single layer of the cultured HBMECs. The P-gp activity of the in-vitro BBB model obtained in the abovementioned case (HBVPs:HAs=1:2) is respectively 82% and 104% higher than the P-gp activity of the BBB model with HBVPs:HAs=1:1 and the P-gp activity of the BBB model with HBVPs:HAs=1:6. Therefore, the in-vitro BBB model with HBVPs:HAs=1:2 can more effectively simulate the in-vivo BBB.

The ratio of HBVPs:HAs=1:1 is deviated from the normal physiological status. In comparison with the BBB model with HBVPs:HAs=1:2, the BBB model with HBVPs:HAs=1:1 has lower TEER and higher PI permeability. Further, the VEGF concentration and the MMP-9 concentration (the concentrations of the inflammatory factors) detected in the BBB model with HBVPs:HAs-1:1 are respectively 1.4 times and 2.1 times higher than these detected in the BBB model with HBVPs:HAs=1:2. Thanabalasundaram, et al. proposed a prior-art co-culture model of rat BMECs and rat BVPs. The Thanabalasundaram model has lower TEER, higher PI permeability, and higher concentrations of inflammatory factors (such as MMPs and VEGF) than those of another prior-art single-layer rat BBB model. The researchers attribute the result of Thanabalasundaram to that the Thanabalasundaram BBB model simulates an inflammation state. Both the model with HBVPs:HAs=1:1 and the prior-art model of Thanabalasundaram, et al. are more close to the normal physiological state, i.e. an excessive ratio of HBVPs cover the surface of HBMECs. Therefore, it is inferred that the co-culture BBB model with HBVPs:HAs=1:1 simulates an inflammatory state.

The ratio of HBVPs:HAs=1:6 is lower than the normal physiological ratio. In comparison with the BBB model with HBVPs:HAs-1:2, the BBB model with HBVPs:HAs=1:6 has lower TEER and higher PI permeability. Further, the VEGF concentration and the MMP-9 concentration (the concentrations of the inflammatory factors) detected in the BBB model with HBVPs:HAs=1:6 are respectively 1.2 times and 4.6 times higher than these detected in with the BBB model with HBVPs:HAs=1:2. From the results of the research teams of Dore-Duffy and Gonul, it is found: in a traumatic or anoxic state, the FIB VPs adhering to HBMECs are likely to emigrate, and the coverage ratio of HBVPs on HBMECs decreases from 1:5 to 1:10-12. Further, the immobile HBVPs are likely to deteriorate in such a case. The experimental results of the present invention suggest that the BBB model with HBVPs:HAs=1:6 may also be regarded as to simulate an inflammatory state and that the BBB model wherein HBVPs and HAs by a ratio of 1:2 are co-cultured with HBMECs, PCM and ACM is more likely to match the normal physiological state.

Below is described the pretreatment of the culture plate. Firstly, add 4 ml of a fibronectin or gelatin solution to the culture plate; next, spread the solution evenly; next, place the culture plate in a carbon-dioxide incubator having a temperature of 37° C. and a relative humidity of 95% for 1 day. Below is also described the pretreatment of the culture dishes. Firstly use disinfected stainless steel forceps to place the culture dishes in a 24-well plate; next, add 500 μL of a fibronectin or gelatin solution to the interior and exterior of each well; next, evenly spread the solution on the filter membranes of the culture dishes; next, place the well plate in a carbon-dioxide incubator having a temperature of 37° C. and a relative humidity of 95% for 1 day.

Below is described the fabrication of the condition mediums used in the present invention. In the fabrication of PCM, plant HBVPs in the pretreated culture plate at a planting density of 4×10⁵ cells/cm² in a carbon-dioxide incubator having a temperature of 37° C. and a relative humidity of 95%, and replace the culture medium each day; while HBVPs have occupied 80% of the culture plate, collect the culture medium, which has been used in cultivation for 1 or 2 days, to function as the so-called pericyte condition medium (PCM₁ or PCM₂). The culture medium may be replaced to repeat the abovementioned process according to a desired interval of time The PCM is collected with a disinfected filter having a porosity diameter of 0.2 μm and stored in a refrigerator at a temperature of −80° C.

In the fabrication of ACM, plant HAs in the pretreated culture plate at a planting density of 4×10⁵ cells/cm² in a carbon-dioxide incubator having a temperature of 37° C. and a relative humidity of 95%, and replace the culture medium each day; while HAs have occupied 80% of the culture plate, collect the culture medium, which has been used in cultivation for 1 or 2 days, to function as the so-called astrocyte condition medium (ACM₁ or ACM₂). The culture medium may be replaced to repeat the abovementioned process according to a desired interval of time. The ACM is collected with a disinfected filter having a porosity diameter of 0.2 μm and stored in a refrigerator at a temperature of −80° C.

In conclusion, the present invention uses different coverage ratios to construct in-vitro human BBB model, which is very similar to the in-vivo human BBB and very helpful to medical research.

The embodiments described above are only to exemplify the present invention but not to limit the scope of the present invention. Any equivalent modification or variation according to the spirit or characteristics of the present invention is to be also included within the scope of the present invention.

What is claimed is:

1. A method to construct an in-vitro human blood brain barrier model, comprising steps:
attaching suspension liquids of human brain vascular pericytes (HBVPs) and human astrocytes (HAs) by a ratio of 1:1, 1:2, or 1:6 to a bottom surface of a filter membrane of a culture dish to plant said HBVPs and said HAs on said bottom surface;
filling a suspension liquid of human brain microvascular endothelial cells (HBMECs) into a top surface of said filter membrane to plant said HBMECs on said top surface;
placing said culture dish in a well plate containing a culture medium, and placing said well plate in a carbon-dioxide incubator;
replacing said culture medium with a condition medium; and
replacing said condition medium once daily for a plurality of days.

2. The method to construct the in-vitro human blood brain barrier model according to claim 1, wherein a planting density of said HBMECs is 4×10⁵ cells/cm². and wherein a total planting density of said HBVPs and said HAs is 4×10⁵ cells/cm².

3. The method to construct the in-vitro human blood brain barrier model according to claim 1, wherein said filter membrane is made of polyethylene terephthalate (PET).

4. The method to construct the in-vitro human blood brain barrier model according to claim 1, wherein said carbon-dioxide incubator has a temperature of 37° C. and a relative humidity of 95%.

5. The method to construct the in-vitro human blood brain barrier model according to claim 1, wherein said condition medium is replaced once daily for 7 days.

6. The method to construct the in-vitro human blood brain barrier model according to claim 1, wherein said culture medium includes an endothelial cell medium, a pericytes culture medium and an astrocyte medium.

7. The method to construct the in-vitro human blood brain barrier model according to claim 1, wherein said condition medium is a pericyte-conditioned medium having been used for 1 day (PCM₁), an pericyte-conditioned medium having been used for 2 days (PCM₂), an astrocyte-conditioned medium having been used for 1 day (ACM), and an astrocyte-conditioned medium having been used for 2 days (ACM₂), or a condition medium containing PCM₂ and ACM₂ by a ratio of 1:1.

8. The method to construct the in-vitro human blood brain barrier model according to claim 1, wherein a liquid level of said culture dish is as high as a liquid level of said culture medium.

9. The method to construct the in-vitro human blood brain barrier model according to claim 1, Wherein said suspension liquids of said HBVPs and said HAs are attached to said bottom for 1 hour.

10. The method to construct the in-vitro human blood brain barrier model according to claim 1, wherein said culture medium is replaced with a condition medium while said HBMECs, said HBVPs and said HAs have occupied 80% of said filter membrane.

11. The method to construct the in-vitro human blood brain barrier model according to claim 1, wherein said suspension liquids of said HBMECs, said HBVPs and said HAs are prepared with a method including steps:
defrosting a plurality of small tubes of frozen liquids respectively containing said HBMECs, said HBVPs and said HAs until said frozen liquids have liquefied completely to form said suspension liquids of said HBMECs, said HBVPs and said HAs; and
respectively sucking said suspension liquids of said HBMECs, said HBVPs and said HAs from said small tubes, and respectively placing said suspension liquids of said HBMECs, said HBVPs and said HAs in culture plates.
